(19) United States
(12) Patent Application Publication
(10) Pub. No.: US 2023/0107746 A1
(43) Pub. Date: Apr. 6, 2023

(54) A Medical Device

(71) Applicant: RIIXO LIMITED, Southport (GB)

(72) Inventor: Cameron JOHNSTON, St Andrews (GB)

(73) Assignee: RIIXO LIMITED, Southport (GB)

(21) Appl. No.: 17/797,710

(22) PCT Filed: Jan. 15, 2021
(86) PCT No.: PCT/GB2021/050097

§ 371 Date: Aug. 4, 2022

(30) Foreign Application Priority Data
Jan. 15, 2020 (GB) 2000620.1

(51) Int. Cl. A61F 7/02

(52) U.S. Cl. CPC A61F 7/02; A61F 2007/0219; A61F 2007/0295; A61F 2007/023; A61F 2007/0223

(57) ABSTRACT

A medical device (10) is provided. The device comprises an elasticated textile body (12) having at least one opening (14,16) into which a limb may be inserted. The device further comprises at least one textile receptacle (17) welded to the elasticated textile body (12), the at least one receptacle containing a thermal storage medium.

### FIELD OF THE INVENTION

The present invention relates to the field of medical devices. More specifically, the present invention is a device which is placed on a joint or limb of the body for treating an area of that joint or limb by applying compression, heat or cold thereto.

### BACKGROUND OF THE INVENTION

It is known that applying compression along with either cold or heat to a particular area of the human body can aid recovery. Applying heat can relax muscles, improve joint mobility and promote tissue healing. Cold can be applied to reduce pain, swelling and muscle soreness. Compression manages swelling whilst optimising oxygen and nutrient delivery for faster recovery, and also provides support.

Compression-applying devices exist which simultaneously allow a person to also apply heat or cold to specific areas of the body. Typically such devices are formed from an elasticated sleeve formed from neoprene or the like, where the device will have one or more pockets sewn into the sleeve. Those pockets receive a gel pack of a known type, which has either been stored in a freezer or heated in a microwave oven depending upon whether the user wishes to apply cold or heat to the area in question.

Having pockets sewn into the sleeve forms seams which can cause discomfort and irritation to the user, and the seams can fail after prolonged use. Additionally, having pockets means that the removable gel pads can shift during use, meaning that the pads may not be applying the desired heat or cold to the optimal area. Furthermore, removable pads may get misplaced resulting in the sleeve becoming nothing more than a conventional compression or support sleeve unless additional pads are purchased by the user.

It is an aim of the present invention to obviate or mitigate one or more of the aforementioned disadvantages.

### SUMMARY OF THE INVENTION

According to the present invention there is provided a medical device comprising:

an elasticated textile body having at least one opening into which a limb may be inserted; and

at least one textile receptacle welded to the elasticated textile body, the at least one receptacle containing a thermal storage medium.

A “thermal storage medium” is a medium which can receive and store thermal energy by being either heated or cooled.

The at least one textile receptacle may be welded to an internal surface of the body.

The body may comprise an inner textile layer and an outer textile layer, wherein the at least one textile receptacle is welded to the inner textile layer.

The outer textile layer may comprise a polyamide and elastic. The outer textile layer may further comprise wool.

The inner textile layer may comprise cotton. The inner textile layer may further comprise elastic.

The thermal storage medium may be a gel. The gel may comprise water and propylene glycol.

The receptacle may be formed from a polyamide.

The textile body may comprise a waistband adapted to fit around the waist of a wearer.

The textile body may comprise a sleeve which is open at both ends. Alternatively, the textile body may comprise a sleeve which is open at one end and closed at an opposite end.

### BRIEF DESCRIPTION OF THE DRAWINGS

Preferred embodiments of the present invention will now be described, by way of example only, with reference to the following drawings, where:

FIGS. 1A, 1B and 1C are respective side, front and isometric views of a medical device for treatment of a user's thigh;

FIGS. 2A, 2B, 2C and 2D are respective front, rear, side and isometric views of a medical device for treatment of a user's knee;

FIGS. 3A, 3B, 3C and 3D are respective front, side rear and isometric views of a medical device for treatment of a user's forearm;

FIGS. 4A, 4B, 4C and 4D are respective front, rear, side and isometric views of a medical device for treatment of a user's foot;

FIGS. 5A, 5B, 5C and 5D are respective front, side rear and isometric views of a medical device for treatment of a user's calf;

FIGS. 6A, 6B, 6C, 6D and 6E are respective front, rear, side, bottom and isometric views of a medical device for treatment of a stump of an above-knee amputee; and

FIGS. 7A, 7B, 7C and 7D are respective front, rear, bottom and isometric views of a medical device for treatment of a stump of a below-knee amputee.

### DETAILED DESCRIPTION OF THE DRAWINGS

In each of the figures the devices are shown turned inside out for ease of viewing the components thereof.

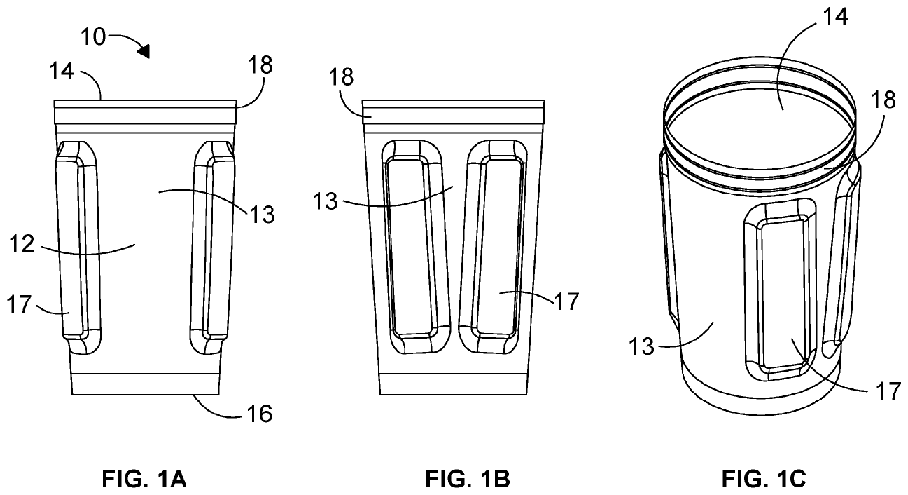

FIGS. 1A, 1B and 1C are respective side, front and isometric views of a medical device for treatment of a user's thigh. The device 10 comprises an elasticated textile body 12 having at least one opening into which a limb may be inserted. In the form shown in FIG. 1 the textile body 12 is a sleeve which has openings 14,16 at either end of the sleeve. The body 12 may also be provided with ribbing 18 at one or both ends thereof.

The body of each and every embodiment of the device described herein may comprise a woven textile formed from a polyamide and elastic. The textile layer may further comprise wool. In a preferred example, the textile may comprise 80% polyamide, 14% elastic and 6% wool. The textile may be waterproof. The textile layer may further comprise bamboo fibre in addition to, or as an alternative to, wool.

The body of each and every embodiment of the device described herein may comprise an inner textile layer and an outer textile layer, where the outer textile layer is as described above. The inner textile layer may comprise cotton. The inner textile layer may further comprise elastic. In a preferred embodiment the inner textile layer may comprise 95% cotton and 5% elastic. Alternatively, the inner textile layer may comprise terry cotton.

A plurality of textile receptacles or bags 17 are provided on an inner surface 13 of the body 12. The receptacles may be formed from nylon or a similar polymeric textile. Contained within each receptacle 17 is a thermal storage medium which can receive and store thermal energy by being either heated (e.g. in a microwave oven) or cooled (e.g. in a freezer). The thermal storage medium may be a gel or rice, for example. One preferred example of the storage medium comprises 60-70% water, 20-35% propylene glycol and up to 5% sodium carboxymethyl cellulose.

The, or each, receptacle 17 is welded to the inner surface 13 or inner layer of the body 12, the inner surface being visible in FIG. 1 due to the device being shown inside out. The textiles are preferably welded to one another using a method selected from the group comprising high frequency welding, hot air welding and hot wedge welding. This means that the, or each, pocket of thermal storage medium is permanently attached to the textile body of the device.

In the thigh treatment device of FIG. 1, there are four receptacles 17 attached to the body 12, and each receptacle has a generally oblong shape. There are two receptacles arranged towards the front of the device and two receptacles arranged towards the rear.

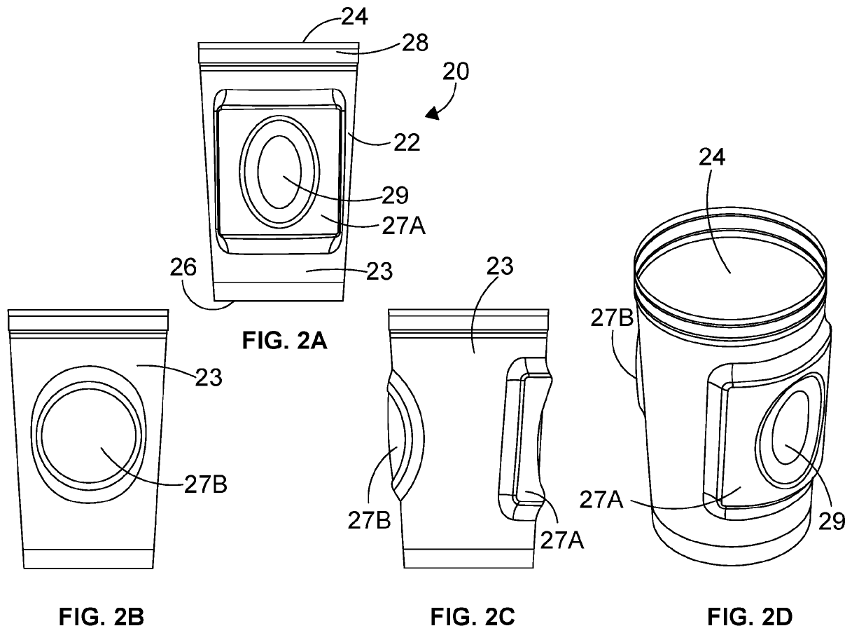

The device shown in FIG. 2 is similar to that shown in FIG. 1, but is intended to fit over the knee of the user as opposed to the thigh. The device 20 comprises an elasticated textile body 22 having at least one opening into which a limb may be inserted. In the form shown in FIG. 2 the textile body 22 is a sleeve which has openings 24,26 at either end of the sleeve. The body 22 may also be provided with ribbing 28 at one or both ends thereof.

A plurality of textile receptacles or bags 27 are provided on an inner surface 23 of the body 22, the device again being inside out in FIG. 2. The receptacles may be formed in the same way as the FIG. 1 device and have the same thermal storage medium contents. The, or each, receptacle 27 is welded to the inner surface 23 or inner layer of the body 22 in the same way as the FIG. 1 device.

In the knee treatment device of FIG. 2, there are a pair of receptacles 27A,27B attached to the body 22. A front receptacle 27A has a generally square shape and an aperture 29 through the middle thereof, with the front receptacle intended to fit over the kneecap of the user. A rear receptacle 27B of the pair is generally circular and is intended to locate behind the knee of the user.

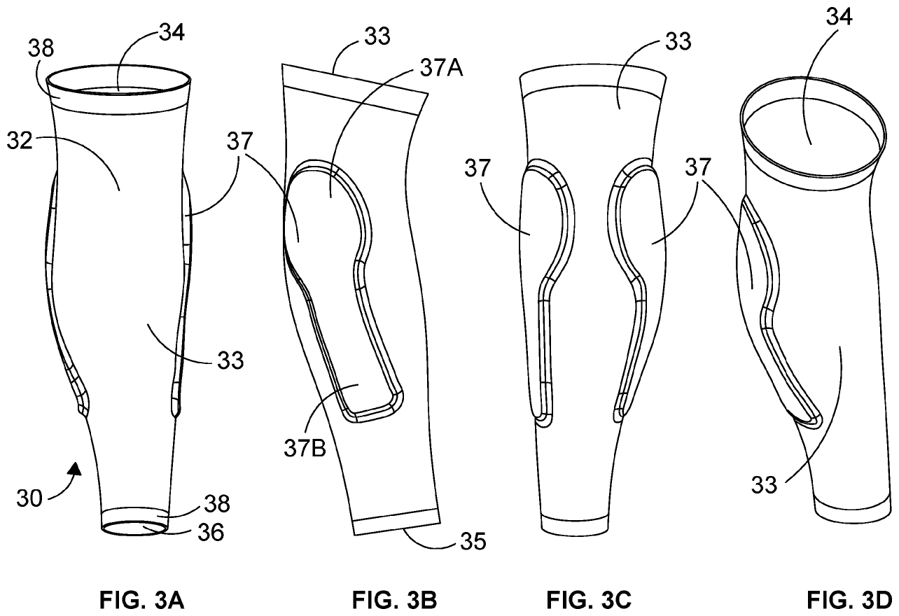

The device shown in FIG. 3 is again similar to those shown in FIGS. 1 and 2, but is intended to fit over the forearm of the user. The device 30 comprises an elasticated textile body 32 having at least one opening into which a limb may be inserted. In the form shown in FIG. 3 the textile body 32 is again a sleeve which has openings 34,36 at either end. The body 32 may also be provided with ribbing 38 at one or both ends thereof.

A plurality of textile receptacles or bags 37 are provided on an inner surface 33 of the body 32, the device again being inside out in FIG. 3. The receptacles may be formed in the same way as the FIGS. 1 and 2 devices and have the same thermal storage medium contents. The, or each, receptacle 37 is welded to the inner surface 33 or inner layer of the body 32 in the same way as described above.

In the forearm treatment device of FIG. 3, there are a pair of receptacles 37 attached to the body 32. Each receptacle 37 has a generally teardrop or keyhole shape. In other words, each receptacle 37 has a first generally circular portion 37A located towards an elbow end 33 of the body 32 and an integral elongated oblong or inwardly tapering portion 37B extending towards a wrist end 35 of the body 32. The receptacles are arranged towards the underside of the body 32 so as to in use lie over the flexor muscles of the forearm.

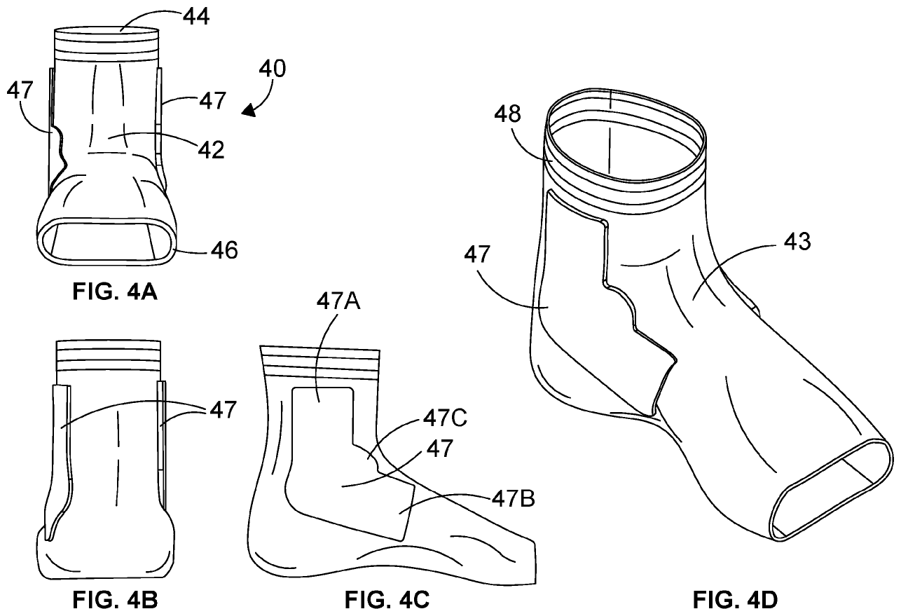

The device shown in FIG. 4 is intended to fit over the foot, including the ankle bone or talus. The device 40 comprises an elasticated textile body 42 which is a generally L-shaped sleeve which has openings 44,46 at either end of the sleeve. The body 42 may also be provided with ribbing 48 at one or both ends thereof.

A pair of textile receptacles or bags 47 are provided on an inner surface 43 of the body 42, the device once again being inside out in FIG. 4. The receptacles may be formed in the same way as the FIG. 1 device and have the same thermal storage medium contents. Each receptacle 47 is welded to the inner surface 43 or inner layer of the body 42 in the same way as the FIG. 1 device.

In the foot treatment device of FIG. 4, the pair of receptacles 47 are attached to either side of the body 22, such that they will lie over either side of a user's ankle bone when in use. The receptacles 47 themselves are generally L-shaped in a similar manner to the body 42. Each receptacle 47 has a first elongate portion 47A which lies generally perpendicular to a second elongate portion 47B. Each receptacle also includes an intermediate portion 47C between the two elongate portions 47A,47B, where the intermediate portion 47C has a part-circular shape.

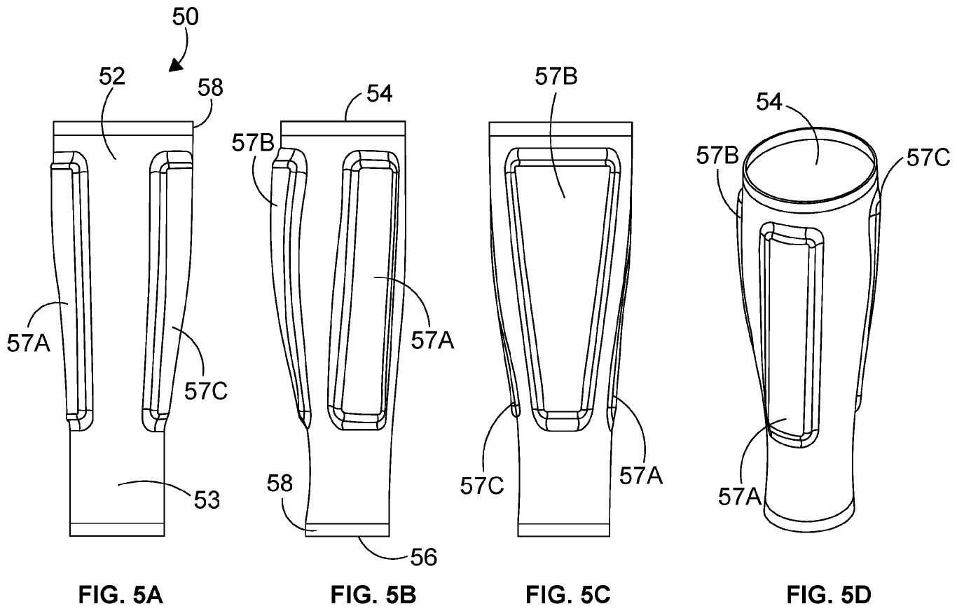

The device shown in FIG. 5 is again similar to those shown in FIGS. 1-3 at least, but is intended to fit over the calf of the user. The device 50 comprises an elasticated textile body 52 having at least one opening into which a limb may be inserted. In the form shown in FIG. 5 the textile body 52 is again a sleeve which has openings 54,56 at either end. The body 52 may also be provided with ribbing 58 at one or both ends thereof.

A plurality of textile receptacles or bags 57 are provided on an inner surface 53 of the body 52. The receptacles may be formed in the same way as the FIGS. 1 and 2 devices and have the same thermal storage medium contents. The, or each, receptacle 57 is welded to the inner surface 53 or inner layer of the body 52 in the same way as described above.

In the calf treatment device of FIG. 5, there are a trio of receptacles 57A,57B,57C attached to the body 52. A central receptacle 57B has a tapered shape, where the lower end of the receptacle 57B is narrower than the top end. The central receptacle 57B is flanked on either side by a pair of outer receptacles 57A,57C. These outer receptacles each have a generally oblong shape. The receptacles are arranged such that the central receptacle 57B will lie over the rear of the calf in use, with the two outer receptacles either side thereof.

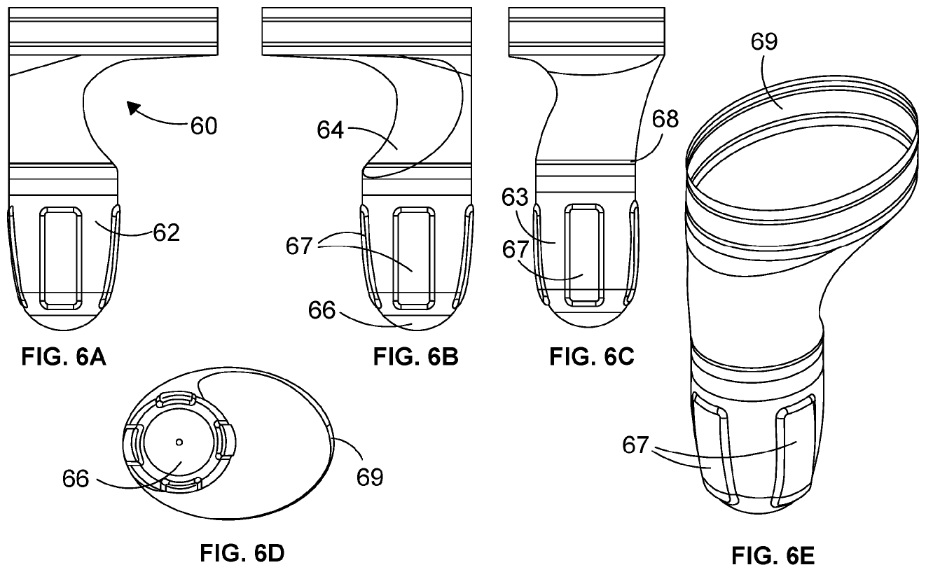

FIGS. 6A-6E are views of a medical device for treatment of the residual limb, or stump, of a user who has had a leg amputation above the knee. The device 60 comprises an elasticated textile body 62 having an opening 64 to receive the stump and a closed lower end 66. The body 62 may also be provided with ribbing 68 at one or both ends thereof.

The body is formed from a textile of the same type as described above, and again may comprise an inner textile layer and an outer textile layer as also described above.

A plurality of textile receptacles or bags 67 are provided on an inner surface 63 of the body 62. The receptacles may be formed from nylon or a similar polymeric textile. Contained within each receptacle 67 is a thermal storage medium of the type described above, which can receive and store thermal energy by being either heated or cooled. Each receptacle 67 is welded to the inner surface 63 or inner layer of the body 62, the inner surface again being visible to the outside in FIG. 6 due to the device being shown inside out. The textiles are preferably welded to one another using one of the methods described above.

In the device of FIG. 6, there are four receptacles 67 attached to the body 62, and these are equidistantly spaced around the body 62. Each receptacle 67 has a generally oblong shape.

An upper end of the body 62 is attached to a waistband 69 adapted to fit around the waist of the user. The waistband 69 may be manually adjustable or alternatively may be elasticated.

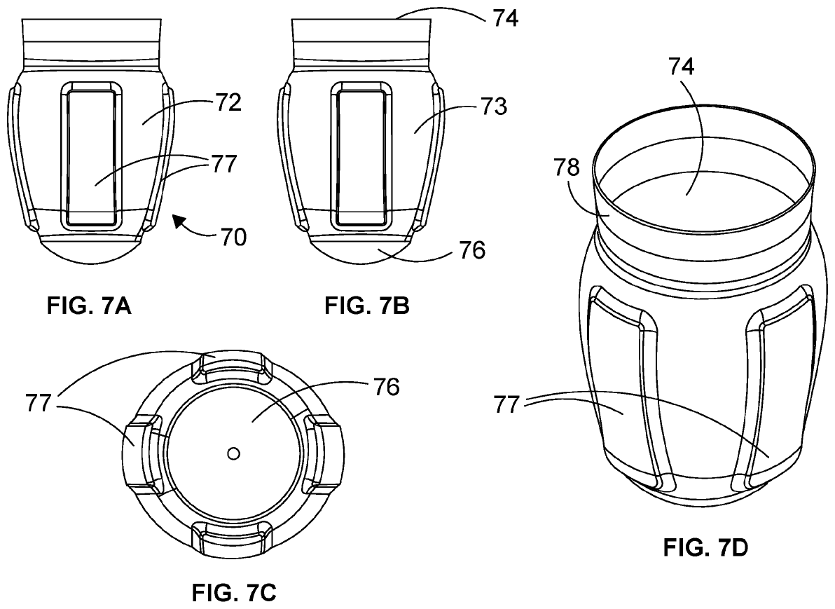

FIGS. 7A-7D are views of a medical device for treatment of the residual limb, or stump, of a user who has had a leg amputation below the knee. The device 70 comprises an elasticated textile body 72 having an opening 74 to receive the stump and a closed lower end 76. The body 72 may also be provided with ribbing 78 at one or both ends thereof.

The body is formed from a textile of the same type as described above, and again may comprise an inner textile layer and an outer textile layer as also described above.

A plurality of textile receptacles or bags 77 are provided on an inner surface 73 of the body 72. The receptacles may be formed from nylon or a similar polymeric textile. Contained within each receptacle 77 is a thermal storage medium of the type described above, which can receive and store thermal energy by being either heated or cooled. Each receptacle 77 is welded to the inner surface 73 or inner layer of the body 72, the inner surface again being visible to the outside in FIG. 7. The textiles are preferably welded to one another using one of the methods described above.

In the device of FIG. 7, there are four receptacles 77 attached to the body 72, and these are equidistantly spaced around the body 72. Each receptacle 77 has a generally oblong shape.

When in use, each of the devices shown in FIGS. 1-7 works in substantially the same way. The elasticated body of the device provides support as well as dynamic gradient compression to the targeted area. This manages any swelling and at the same time optimises oxygen and nutrient delivery to the area.

Depending upon whether the device is to deliver heat or cold to the area, it can be heated in a heat source such as a microwave oven or else chilled in a freezer. If the device is heated the thermal storage medium in the receptacle(s) delivers radiating heat to the area, relaxing muscles to improve joint mobility whilst also promoting tissue healing. If the device is chilled the thermal storage medium delivers cold to the area, which reduces pain, swelling and muscle soreness.

By welding the receptacles containing the thermal storage medium to the body of the device, the present invention ensures that there are no seams present, thus avoiding discomfort and irritation to the user. Additionally, given that the receptacles are permanently welded to the body means that the thermal storage medium is optimally positioned to treat the muscle groups, ligaments and tendons of a given area of the body, with no risk that the medium can shift position during use. Furthermore, the medium cannot get misplaced.

The receptacles used to house the thermal storage medium may be circular, oblong or tapered, where one end is narrower than an opposite end. The receptacles may also be kidney-, keyhole- or teardrop-shaped. The receptacles may have at least one aperture extending through the receptacle, in the manner described above in relation to the knee treatment device.

Instead of the preferred thermal storage media described above, the present invention may instead use clay, beads or gel pearls as the thermal storage medium.

Whilst it is preferred that each receptacle is welded to an internal surface of the body, it is also possible to weld each receptacle to an outer surface of the body in particular applications.

Whilst it is preferred that the primary component of the body is a polyamide, other polymeric compounds may also be used instead such as, for example, polyacrylonitrile (PAN) or polyvinyl chloride (PVC).

The body of each device may be formed using a circular or flat knitting technique.

Modifications and improvements may be incorporated without departing from the scope of the present invention.

1. A medical device comprising:
an elasticated textile body having at least one opening into which a limb may be inserted; and
at least one textile receptacle welded to the elasticated textile body, the at least one receptacle containing a thermal storage medium.

2. The medical device of claim 1, wherein the at least one textile receptacle is welded to an internal surface of the body.

3. The medical device of claim 1, wherein the body comprises an inner textile layer and an outer textile layer, and wherein the at least one textile receptacle is welded to the inner textile layer.

4. The medical device of claim 3, wherein the outer textile layer comprises a polyamide and elastic.

5. The medical device of claim 4, wherein the outer textile layer further comprises wool.

6. The medical device of claim 3, wherein the inner textile layer comprises cotton.

7. The medical device of claim 6, wherein the inner textile layer further comprises elastic.

8. The medical device of claim 1, wherein the thermal storage medium is a gel.

9. The medical device of claim 8,wherein the gel comprises water and propylene glycol.

10. The medical device of claim 1, wherein the receptacle is formed from a polyamide.

11. The medical device of claim 1, wherein the textile body comprises a waistband adapted to fit around the waist of a wearer.

12. The medical device of claim 1, wherein the textile body comprises a sleeve which is open at both ends.

13. The medical device of claim 1, wherein the textile body comprises a sleeve which is open at one end and closed at an opposite end.
